(19) United States
(12) Patent Application Publication
(10) Pub. No.: US 2010/0124538 A1
(43) Pub. Date: May 20, 2010

(54) TOPICAL APPLICATION OF FLUOCINOLONE ACETONIDE FOR DEPIGMENTATION OF THE SKIN

(75) Inventors: Isabelle Pelisson, Vallauris (FR); André Jomard, Saint Vallier de Thiey (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 12/541,393

(22) Filed: Aug. 14, 2009

Related U.S. Application Data
(63) Continuation of application No. PCT/EP08/52298, filed on Feb. 26, 2008.
(60) Provisional application No. 60/903,311, filed on Feb. 26, 2007.

(51) Int. Cl. A61K 8/63; A61Q 19/02; A61Q 17/04

(52) U.S. Cl. 424/60; 424/62; 424/59

(57) ABSTRACT

Topically applicable compositions useful for the depigmentation of the skin contain a fluocinolone acetonide depigmenting agent, at a concentration of from 0.0001% to 0.02% by weight, of the total weight of the composition, formulated into physiologically acceptable carrier therefor.

### CROSS-REFERENCE TO PRIORITY/PROVISIONAL APPLICATIONS

This application claims priority under 35 U.S.C. §120 of U.S. Provisional Application No. 60/903,311, filed Feb. 26, 2007, and is a continuation/national phase of PCT/EP 2008/052298, filed Feb. 26, 2008 and designating the United States (published in the English language on Sep. 4, 2008 as WO 2008/104532 A1), each hereby expressly incorporated by reference in its entirety and each assigned to the assignee hereof.

### BACKGROUND OF THE INVENTION

1. Technical Field of the Invention

The present invention relates to topical compositions for depigmenting the skin, comprising fluocinolone acetonide, and to the administration of such compositions in the treatment of pigmentary disorders.

2. Description of Background and/or Related and/or Prior Art

Skin pigmentation results from the synthesis of melanin in the melanocytes. This synthesis takes place in organelles called melanosomes, through the conversion of tyrosine under the influence of a cuproprotein enzyme, tyrosinase. The melanosomes are then transferred to the adjacent keratinocytes via the melanocyte dendrites. The keratinocytes thus pigmented begin their differentiation process to the surface of the skin.

A large number of pigmentation disorders associated with excessive or unwanted production of melanin exists. By way of example, melasma is a condition which is characterized by the appearance of dark spots on the cheeks, the forehead and sometimes the lips and the neck. This condition generally appears during pregnancy (melasma gravidarum or “the mask of pregnancy”), at the menopause when taking hormone replacement therapy, or when taking contraceptive pills. This pigmentation disorder is encountered more rarely in men, who represent only 10% of cases. The precise cause of melasma remains unknown; multiple aetiopathogenic factors have nevertheless been mentioned, the most important being genetic predisposition, exposure to sunlight and oestrogens (mainly pregnancy and contraceptives).

Other examples of pigmentary disorders include age spots (lentigo senilis), or, alternatively, hyperpigmentations which appear following an inflammation of the skin (inflammatory dermatosis, irritant drug treatment, irritant procedure, cicatricial process).

A large number of plant extracts and compounds are reported as having activity against hyperpigmentation, in particular ascorbic acid and its derivatives, kojic acid and its derivatives and extract of liquorice (glycyrrhiza). However, these compounds and extracts are moderately active in the treatment and prevention of pigmentary disorders.

Several combinations of active agents have also been tested with greater or lesser success. From the publication by Kligman and Willis, Arch. Dermatol., vol. 111, January 1975, p. 40-48, a formulation comprising 0.1% of tretinoin, 5% of hydroquinone and 0.1% of a topical corticosteroid, dexamethasone, is described. This composition makes it possible to obtain a complete depigmentation of the skin after treatment for 5 to 7 weeks. On the other hand, no depigmentation is obtained when one of the three components is omitted, although it would have been advantageous to have a monotherapy or a bitherapy in order to limit as much as possible the many side effects associated with these three types of components.

One example of monotherapy consists of the administration of a composition comprising hydroquinone, the latter having long been known for its depigmenting activity. However, its use in the long term or at high concentration is associated with cutaneous side effects: irritation, contact allergy, post-inflammatory hyperpigmentation, or even definitive depigmentation or ochronosis.

Tretinoin, or retinoic acid, has a recognized but limited depigmenting effect in melasma, and its mechanism of action in this indication is poorly understood. It is thought to bring about a decrease in epidermal melanin through an increase in cell renewal and perhaps also through inhibition of tyrosinase (Griffiths et al., J Dermatol., vol. 129 1993, p. 415-421). However, tretinoin treatment causes adverse effects (erythema, irritation, desquamation) and the time necessary to obtain a significant result is three times longer than with hydroquinone alone (40 weeks versus 12 weeks with hydroquinone).

With limited effectiveness and considerable side effects, the depigmenting agents used at the current time are not satisfactory and there exists a need for an effective treatment for hyperpigmentation disorders with few side effects.

The treatment of hyperpigmentation with corticosteroids has shown that dexamethasone alone does not induce any depigmentation (publication by Kligman and Willis, Arch. Dermatol., vol. 111, January 1975, p. 40-48). Another publication has shown a certain depigmenting effect of clobetasol propionate, which is a powerful corticosteroid (Kanwar et al., Dermatology 1994, p. 188:170): a lightening of 80% to 90% of the pigmentation was obtained after 6 to 8 weeks of topical treatment with clobetasol propionate at 0.05%, in patients suffering from melasma. However, in 30% of cases, the treatment had to be stopped after 4 weeks due to the appearance of local atrophy and of telangiectasiae. In addition, for 57% of the patients in whom a lightening of the pigmentation was observed, the melasma reappeared at the same sites 2 to 3 weeks after the treatment had been stopped, and gradually returned to its initial state after 4 to 6 months.

The appearance of such important side effects and the transient lightening phenomenon observed may be attributed to the fact that clobetasol propionate is a powerful corticosteroid and that it was used at high concentration. These reasons led the assignee hereof to develop a novel topical composition comprising another corticosteroid and especially at very low concentration, while at the same time conserving long-term depigmenting properties.

### SUMMARY OF THE INVENTION

Thus, it has now been demonstrated, surprisingly, that fluocinolone acetonide administered at very low concentrations elicits considerable depigmenting activity.

Indeed, it has now been demonstrated that fluocinolone acetonide makes it possible to obtain rapid and effective depigmentation from the beginning of treatment onwards at very low doses, from the concentration of 0.0001% by weight, of the total weight of the composition, onwards. At low doses, fluocinolone acetonide makes it possible to obtain a depigmenting activity that is greater and earlier than that obtained with other corticosteroids such as hydrocortisone, desonide and 13-methasone valerate.

In addition, all these depigmenting activities of fluocinolone acetonide are accompanied by very limited side effects.

Given the above, this invention features topical compositions containing fluocinolone acetonide at low concentrations, that are useful for treating and/or preventing skin hyperpigmentation disorders. These compositions, by virtue of their low concentrations of active ingredient, make it possible to limit the side effects potentially associated with the administration of fluocinolone acetonide.

The first aspect of the invention is thus a topical composition for depigmentation of the skin, comprising fluocinolone acetonide as depigmenting agent and a physiologically acceptable carrier, wherein the concentration of fluocinolone acetonide ranges from 0.0001% to 0.02% by weight, of the total weight of the composition.

The present invention more particularly features topical compositions comprising fluocinolone acetonide, as defined above, but containing neither hydroquinone nor tretinoin.

The second aspect is the formulation of such compositions into medicaments useful in the treatment and/or prevention of skin pigmentation disorders.

The third aspect is the administration of such compositions for whitening the skin and for protecting the skin against the harmful effects of sunlight.

Finally, the fourth aspect is a non-therapeutic cosmetic treatment regime or regimen for embellishing the skin or its surface appearance, in which a composition comprising fluocinolone acetonide as depigmenting agent at a concentration of from 0.0001% to 0.02% by weight of the total weight of the composition and a physiologically acceptable carrier, is topically applied to the skin and/or its integuments.

### BRIEF DESCRIPTION OF THE DRAWINGS

FIG. 1 is a representation in the form of a histogram of a pigmentation score on the tail of SKH:HR2 mice after 4 weeks of topical application of fluocinolone acetonide or of hydrocortisone;

FIG. 2 is a representation in the form of a histogram of the surface area positive for Fontana-Masson staining/μm of epidermis after 4 weeks of topical application of fluocinolone acetonide or of hydrocortisone to the tail of SKH:HR2 mice;

FIG. 3 is a schematic representation of depigmentation kinetics for the tail of SKH:HR2 mice after 5 weeks of topical application of fluocinolone acetonide, of desonide and of p-methasone valerate;

FIG. 4 is a schematic representation of depigmentation kinetics for the tail of SKH:HR2 mice after 4 weeks of topical application of fluocinolone acetonide; and

FIG. 5 is a schematic representation of depigmentation kinetics for the tail of SKH:HR2 mice after 4 weeks of topical application of desonide.

### DETAILED DESCRIPTION OF BEST MODE AND SPECIFIC/PREFERRED EMBODIMENTS OF THE INVENTION

As employed above, and throughout the description of the invention, the following terms, unless otherwise mentioned, should be understood to have the following meanings.

The term “depigmenting agent” means any active agent having a depigmenting activity on the skin. This activity makes it possible to reduce the pigmentation of the skin that already exists and/or also to prevent any additional pigmentation greater than the natural pigmentation.

The term “physiologically acceptable carrier” means, within the scope of a valid medical judgement, a carrier suitable for use in contact with the cells of humans and animals without toxicity, irritation, undue allergic response, and the like.

In the subsequent text, unless otherwise indicated, the proportions of the various constituents of the composition are expressed as percentage by weight (m/m) of the total weight of said composition.

The amount of fluocinolone acetonide may vary within a range of from 0.0001% to 0.02%. Preferably, the compositions according to the invention comprise a concentration of fluocinolone acetonide of from 0.0005% to 0.01%.

The composition according to the invention may also comprise one or more sunscreen(s) in preferred concentrations ranging from 0.001 to 30%. Among the sunscreens, examples are physical sunscreens, such as titanium dioxide or zinc oxide, and chemical sunscreens, such as octocrylene, ethylhexyl methoxycinnamate, octyl salicylate, avobenzone, oxybenzone, ecamsule or drometrizole trisiloxane, taken alone or as mixtures.

The compositions according to the invention may also comprise any additive normally used in the cosmetics or pharmaceutical field, such as sequestering agents, antioxidants, preservatives, fillers, electrolytes, humectants, dyes, common inorganic or organic bases or acids, fragrances, essential oils, active cosmetic agents, moisturizers, vitamins, essential fatty acids, sphingolipids, self-tanning compounds, and agents for soothing and protecting the skin.

Of course, those skilled in the art will take care to select this or these optional additional compounds) and/or the amount thereof in such a way that the advantageous properties of the compositions according to the invention are not, or are not substantially, impaired.

These additives may be present in the composition in a proportion of from 0.001% to 20%.

The compositions according to the invention are preferably for use in dermatology. They may therefore be administered as a medicament. They may also be applied as a cosmetic product.

The compositions according to the invention may be in any of the galenical forms normally used for topical application, for example in the form of solutions, gels, dispersions of the lotion or serum type, emulsions having a liquid or semi-liquid consistency of the milk type, obtained by dispersion of a fatty phase in an aqueous phase (O/W) or conversely (W/O), or suspensions or emulsions having a soft, semi-solid or solid consistency of the cream or gel type, or else microemulsions, microcapsules, microparticles or vesicular dispersions of ionic and/or non-ionic type. These compositions are prepared according to the usual methods.

In a known manner, the pharmaceutical or cosmetic compositions of the invention may contain adjuvants common in the cosmetics or dermatological field, such as emulsifiers, hydrophilic or lipophilic gelling agents, hydrophilic or lipophilic active agents, preservatives, antioxidants, fragrances, fillers, screening agents and dyestuffs. The amounts of these various adjuvants are those conventionally employed in the cosmetics and/or dermatological fields and will be from 0.01% to 20% of the total weight of the composition. Depending on their nature, these adjuvants may be introduced into the fatty phase, into the aqueous phase and/or into the lipid vesicles.

According to a second embodiment of the invention, the subject compositions are formulated into medicaments for use in the treatment and/or prevention of skin pigmentation disorders.

According to a third embodiment, the compositions according to the invention are used in the cosmetics field, in particular for whitening the skin, or else for protection against the harmful effects of sunlight.

The term “skin pigmentation disorders” means disorders such as melasma, chloasma, lentigines, senile lentigo, post-inflammatory hyperpigmentations due to abrasion and/or burns and/or scars and/or dermatosis and/or contact allergy and/or irritant treatment; freckles, ephelides, planar pigmented seborrhoeic warts, naevi, genetically determined hyperpigmentations, hyperpigmentations of metabolic or drug-related origin, or any other hyperpigmentary lesions, in particular those induced by photo-induced or chronological aging of the skin and of the integuments.

According to a fourth embodiment, a non-therapeutic cosmetic treatment regime or regimen is provided for embellishing the skin or its surface appearance, in which a composition comprising fluocinolone acetonide as depigmenting agent at a concentration of from 0.0001% to 0.02% by weight of the total weight of the composition and a physiologically acceptable carrier, is applied to the skin and/or its integuments.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Example 1

### Comparative Evaluation of the Depigmenting Activity of Fluocinolone Acetonide and of Hydrocortisone Applied Topically for 4 Weeks to the Tail of SKH:HR2 Mice

The proportions of the various constituents are expressed as a percentage and by weight relative to the total weight of the composition.

Materials and Methods:

The depigmenting activity of fluocinolone acetonide and of hydrocortisone was evaluated on the tail of female SKH:HR2 mice that were 6 weeks old at the beginning of the study. The products were applied topically (20 μl of product to the tail), 5 days a week for 4 weeks. Each group contains 5 animals:

Group 1: carrier control (acetone)

Group 2: fluocinolone acetonide at 0.0005%

Group 3: fluocinolone acetonide at 0.01%

Group 4: hydrocortisone at 0.01%

Group 5: hydrocortisone at 0.05%

Group 6: hydrocortisone at 0.1%

Group 7: hydrocortisone at 0.5%.

Evaluation Methods:

Clinical observations: once a week the pigmentation is scored on a scale ranging from 0 (base pigmentation) to −4 (total depigmentation).

A tail skin sample is taken at the end of the study and fixed with formol in order to carry out a histological analysis.

The epidermal surface area taken up by melanin is measured by image analysis on histological sections stained by the Fontana-Masson technique, which makes it possible to reveal the pigments. For each animal, the Fontana-Masson-positive surface area is measured by image analysis on five fields of interfollicular epidermis and weighted with respect to the length of corresponding epidermis in order to avoid any possible bias due to hyperplasia or atrophy of the epidermis. Since the horny layer is partially lost during the treatment of the samples, the measurement is carried out only in the living layers of the epidermis.

Results:

Clinical Scores on the Tail of SKH:HR2 Mice:

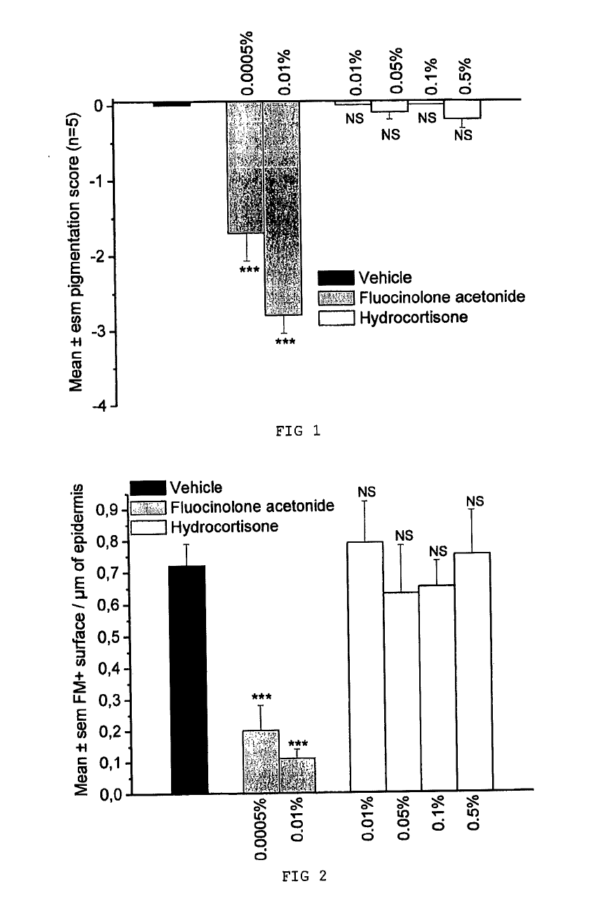

The results obtained are reported in FIG. 1: pigmentation scores on the tail of SKH:HR2 mice treated with fluocinolone acetonide or hydrocortisone for 4 weeks.

Fluocinolone acetonide at 0.01% results in marked depigmentation of the tail after 4 weeks of treatment, compared with hydrocortisone at the same concentration. The depigmenting activity of fluocinolone acetonide remains substantial, even at a concentration of 0.0005%, whereas hydrocortisone has only a very weak depigmenting activity, even when the concentrations are increased to 0.5%.

Fontana-Masson Staining on the Tail of SKH:HR2 Mice:

The results obtained are illustrated in FIG. 2: Fontana-Masson-positive surface area in the living epidermis of the tail of SKH:HR2 mice treated for 4 weeks with fluocinolone acetonide or hydrocortisone.

After 4 weeks of topical application, it clearly appears that only the fluocinolone acetonide induced a drastic decrease in the Fontana-Masson-positive surface area in the living layers of the tail epidermis. The decrease is 85% at the concentration of 0.01% to 72% at the concentration of 0.0005%. On the other hand, hydrocortisone does not significantly modify the Fontana-Masson-positive surface area after 4 weeks of topical application up to the concentration of 0.5%.

These studies show the specific efficacy of fluocinolone acetonide on skin depigmentation, even at very low concentrations.

### Example 2

### Comparative Evaluation of the Depigmenting Activity of Various Corticosteroids Applied Topically for 5 Weeks to the Tail of SKH:HR2 Mice:

Materials and Methods:

The depigmenting activities of three corticosteroids, fluocinolone acetonide, desonide and β-methasone valerate, were evaluated on the tail of female SKH:HR2 mice that were 8 weeks old at the beginning of the study. The products were applied topically (20 μl of product to the tail), 5 days a week for 5 weeks. Each group contains 6 animals.

Group 1: carrier control (acetone)

Group 2: fluocinolone acetonide at 0.01%

Group 3: desonide at 0.01%

Group 4: p-methasone valerate at 0.01%.

Evaluation Methods:

Clinical observations: once a week, the pigmentation is scored on a scale ranging from 0 (base pigmentation) to −4 (total depigmentation).

Results:

Clinical Scores on the Tail of SKH:HR2 Mice:

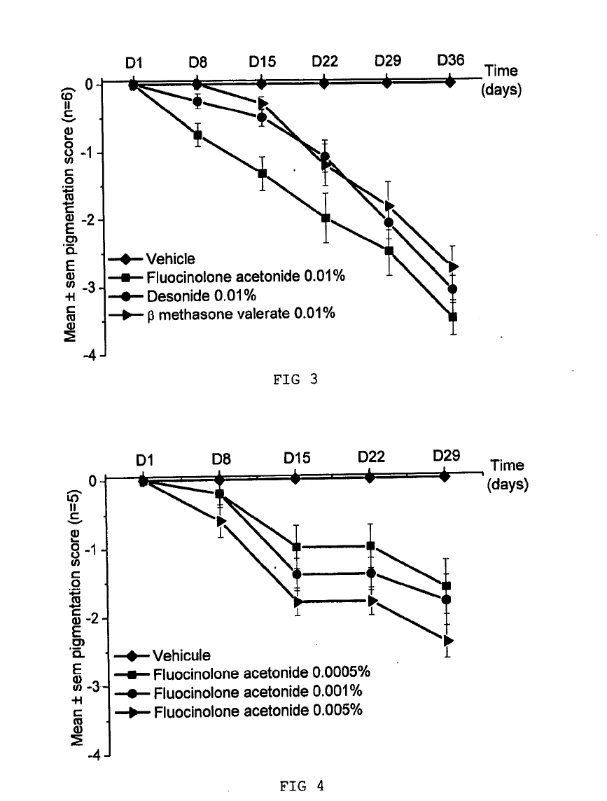

The results obtained are reported in FIG. 3: pigmentation scores on the tail of SKH:HR2 mice treated with fluocinolone acetonide, desonide and β-methasone valerate for 5 weeks.

At an equivalent concentration of 0.01%, fluocinolone acetonide has a greater depigmenting activity than desonide and than β-methasone valerate. The depigmentation observed with fluocinolone acetonide is virtually total after 5 weeks.

### Example 3

### Comparative Evaluation of the Depigmenting Activity of Fluocinolone Acetonide and of Desonide Applied Topically for 4 Weeks to the Tail of SKH:HR2 Mice

Materials and Methods:

The depigmenting activity of fluocinolone acetonide and of desonide was evaluated on the tail of female SKH:HR2 mice that were 8 weeks old at the beginning of the study. The products were applied topically (20 μl of product to the tail), 5 days a week for 4 weeks. Each group contains 5 animals:

Group 1: carrier control (acetone)

Group 2: fluocinolone acetonide at 0.0005%

Group 3: fluocinolone acetonide at 0.001%

Group 4: fluocinolone acetonide at 0.005%

Group 5: desonide at 0.0005%

Group 6: desonide at 0.001%

Group 7: desonide at 0.005%.

Evaluation Methods:

Clinical observations: once a week, the pigmentation is scored on a scale ranging from 0 (base pigmentation) to −4 (total depigmentation).

Results:

Clinical Scores on the Tail of SKH:HR2 Mice:

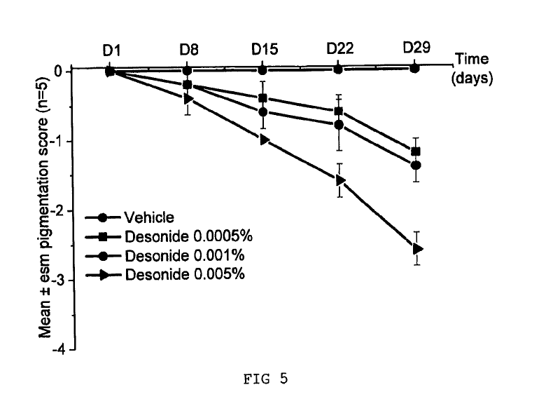

The results obtained are reported in FIGS. 4 and 5: pigmentation scores on the tail of SKH:HR2 mice treated with fluocinolone acetonide (FIG. 4) or desonide (FIG. 5) for 4 weeks.

At a very low concentration of 0.005%, fluocinolone acetonide has a very significant depigmenting activity that is much more rapid (from the 8th day onwards) than desonide at the same concentrations. This depigmenting activity remains greater than that of desonide at an equivalent concentration throughout the study.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable dermatological composition useful for the depigmentation of skin, comprising a fluocinolone acetonide depigmenting agent formulated into a topically applicable, physiologically acceptable carrier therefor, the concentration of said fluocinolone acetonide depigmenting agent ranging from 0.0001% to 0.02% by weight, of the total weight of the composition.

2. The topical dermatological composition as defined by claim 1, comprising neither hydroquinone nor tretinoin.

3. The topical dermatological composition as defined by claim 1, wherein the concentration of fluocinolone acetonide ranges from 0.0005% to 0.01% by weight, of the total weight of the composition.

4. The topical dermatological composition as defined by claim 1, further comprising at least one sunscreen.

5. The topical dermatological composition as defined by claim 1, formulated as a medicament.

6. The topical dermatological composition as defined by claim 1, formulated as a cosmetic product.

7. A regime or regimen for the whitening or depigmenting of the skin, comprising topically applying thereon, for such period of time as required to elicit the desired effect, a thus effective amount of the dermatological composition as defined by claim 1.

8. A regime or regimen for photoprotecting the skin against the damaging effects of sunlight, comprising topically applying thereon, for such period of time as required to elicit the desired effect, a thus effective amount of the dermatological composition as defined by claim 4.

9. A regime or regimen for the treatment and/or prevention of melasma, chloasma, lentigines, senile lentigo, post-inflammatory hyperpigmentations due to abrasion and/or burns and/or scars and/or dermatosis and/or contact allergy and/or an irritant treatment; freckles, ephelides, planar pigmented seborrhoeic warts, naevi, genetically determined hyperpigmentations, hyperpigmentations of metabolic or drug-related origin, other hyperpigmentary lesions, those induced by photo-induced or chronological aging of the skin and of the integuments, comprising topically applying onto the afflicted skin area of a subject affected therewith, for such period of time as required to elicit the desired effect, a thus effective amount of the dermatological composition as defined by claim 1.

10. A regime or regimen for the treatment and/or prevention of melasma, comprising topically applying onto the afflicted skin area of a subject affected therewith, for such period of time as required to elicit the desired effect, a thus effective amount of the dermatological composition as defined by claim 1.

11. A non-therapeutic cosmetic treatment regime or regimen for embellishing the skin or its surface appearance, comprising topically applying to the skin and/or its integuments a composition comprising fluocinolone acetonide as depigmenting agent at a concentration of from 0.0001% to 0.02% by weight of the total weight of the composition and a physiologically acceptable carrier.
